(19) United States
(12) Patent Application Publication
(10) Pub. No.: US 2006/0036167 A1
(43) Pub. Date: Feb. 16, 2006

(54) Vascular image processing

(75) Inventor: Yuval Shina, Givat Ada (IL)

Correspondence Address: WOLF, BLOCK, SCHORR & SOLIS-COHEN LLP, 250 PARK AVENUE, NEW YORK, NY, 10177, US

(73) Assignee: Shina Systems Ltd., Caesarea (IL)

(21) Appl. No.: 11/175,637

(22) Filed: Jul. 5, 2005

Related U.S. Application Data
(60) Provisional application No. 60/585,222, filed on Jul. 3, 2004.

(51) Int. Cl. A61B 6/00

(52) U.S. Cl. 600/433

(57) ABSTRACT

A method of estimating a position of a foreign object in the body, comprising: (a) providing a 3D data set of at least one blood vessel; (b) acquiring at least one 2D projection image of the vessel including the object; (c) registering the 2D projection image of the vessel to the 3D data set; and (d) using the registration to estimate a 3D position of said object restricted to be in a blood vessel, according to said 3D data set.

### RELATED APPLICATIONS

The present invention claims the benefit under 119(e) of U.S. provisional application 60/585,222, filed Jul. 3, 2004, the disclosure of which is incorporated herein by reference.

### FIELD OF THE INVENTION

The present invention relates to vascular data processing, for example, using CT data to enhance the usability of IVUS data and positioning of catheters on an angiography image.

### BACKGROUND OF THE INVENTION

A recent development is IVUS (intra vascular ultrasound) in which an IVUS head of a catheter images a slice perpendicular to the vessel axis, using ultrasound imaging. In use, the catheter is pulled back to obtain multiple slices. However, combining the slices into a complete 3D data set is not easy.

“Registration and Analysis of Vascular Images”, Stephen R. Aylward, Julien Jomier, Sue Weeks, Elizabeth Bullitt and “Retrospective registration of x-ray angiogram with MR images by using vessels as intrinsic landmarks”, Journal of Magnetic Resonance Imaging: 4, 1994, pp. 139-144, the disclosures of which are incorporated herein by reference, describe the registration of angiography CT and MR images to x-ray angiography images using image processing methods.

“Three-dimensional reconstruction of the coronary artery wall by image fusion of intravascular ultrasound and bi-plane angiography”, by Robert M. Cothren, Raj Shekhar, E. Murat Tuzcu, Steven E. Nissen, J. Fredrick Cornhill and D. Geoffrey Vince, International Journal of Cardiac Imaging, 16(2) 69-85, 2000, the disclosures of which are incorporated herein by reference, describe determining a 3D position of an IVUS head based on x-ray angiographic images.

“Accurate 3D reconstruction of complex blood vessel geometries from intravascular ultrasound images: in vitro study”, K. R. Subramanian, M. J. Thubrikar, B. Fowler, M. T. Mostafavi, M. W. Funk, Journal of Medical Engineering & Technology, Volume 24, Number 4, Jul. 1, 2000, pp 131-140, the disclosure of which is incorporated herein by reference, describes using a bi-plane imaging system to extract an approximate 3D vessel structure for IVUS positioning.

One limitation of x-ray angiography is that only the inner lumen of a diseased vessel is visible. For example, outer remodeling of the vessel (bulging) is not visible. Also, the cause of narrowing is not ascertainable, for example, determining if the plaque is fatty or calcified. It is not believed that soft plaque which is badly attached to the vessel wall is a more dangerous plaque than hard plaque which is well attached. Also, it is noted that when plaque first forms, it does not necessarily extend into the lumen a significant amount. For these reasons, diagnosis is preferentially carried out using IVUS, and in some cases, CT imaging, which allow these plaque characteristics to be identified. Another problem caused by soft plaque is that during angiography, soft plaque may be damaged by the catheter (as the physician is not aware it is there), causing various dangerous side effects.

### SUMMARY OF THE INVENTION

An aspect of some embodiments of the invention relates to using a 3D data set during catheterization. In an exemplary embodiment of the invention, a 3D data set, for example a multi-slice CT data set (MSCT), is registered to a single or multi-plane projection image acquired during catheterization and used to generate a 3D position of a catheter or other foreign object (optionally movable) that is inside a body. Alternatively or additionally, data from the 3D data set and from the image are combined, for example, to generate an enhanced presentation or to correct for errors in data.

In an exemplary embodiment of the invention, the position of the catheter is constrained to match a position indicated in said 3D data set to be inside a blood vessel.

In an exemplary embodiment of the invention, the projection image(s) is an angiography image optionally acquired using an ECG gated imager and registered to a CT data set, for example a data set of the heart. In an exemplary embodiment of the invention, a vascular tree of the heart is extracted from the CT data set and optionally used to find a correct transformation that projects the 3D data set onto the projection image. Optionally, the projection image and the data set are registered based on a correspondence of the vascular tree and the angiographic image. The catheter position can be determined responsive to this registration.

In an exemplary embodiment of the invention, the determined catheter position is a position of an IVUS head. Optionally, a series of IVUS images (slices) acquired at a series of such positions are stitched together into a 3D IVUS data set. Optionally, the CT data set is used to assist in stitching together slices acquired by IVUS and/or in correcting IVUS data.

In some embodiments of the invention, the catheter is a treatment catheter, for example, a balloon catheter or a stent delivery catheter.

In an exemplary embodiment of the invention, a position of a catheter tip is estimated by measuring an amount or rate of motion of the catheter and projecting that motion along a vessel as delineated in the 3D data set. The motion and/or speed may then be used to estimate a new position based on the old position and the amount of motion. Optionally, this method is used for forward motion of the catheter as well as or instead of pull-back motion. Optionally, the motion is detected using a detector that is outside the body, for example, a detector associated with a pull-back mechanism.

In an exemplary embodiment of the invention, the tip of the catheter is detected using a tip detection method which does not require an additional injection of contrast material and may use reduced radiation doses. For example, the catheter may include a radio-opaque marker readily visible on a background of tissue.

In an exemplary embodiment of the invention, data from the 3D data set and the image are combined, for example, blended. Such combination can result, for example, in an image or in a new 3D data set. For example, CT data includes information about soft plaque and/or outer modeling, which is not found in angiography. Optionally, data from an IVUS head is combined with the data set and/or projection image, for example, generating a data set including two or more of soft plaque information, calcified plaque information, vessel lumen and/or vessel outer diameter. In an exemplary embodiment of the invention, the data set is shown as slices. Optionally, the data set is shown as virtual IVUS images, even where actual IVUS data was not acquired.

In an exemplary embodiment of the invention, an enhanced angiographic image is produced, in which one or more vascular structures that were extracted from the data set are shown. For example, the image can show soft plaque, outer modeling and/or bifurcations. Optionally, the structures are obtained by segmenting the 3D data set. Optionally, the presentation is used to selectively enhance characteristics of the data. For example, a soft plaque section, once projected onto an angiography image may be near invisible. Optionally, however, the location of soft plaque is marked in a manner that is visible more than the size and/or projected size of the plaque warrants, for example, using a highlight color, using callouts and/or using an arrow or other indication or symbol.

In an exemplary embodiment of the invention, the better structural definition in a 3D data set (such as CT) is used to complement better spatial resolution but otherwise limited data sets, for example, as in IVUS (where much data may be missing and/or be at an unknown position) and/or angiographic images (where the projection hides structural information). Optionally, it is the segmentation of the 3D data set which is used to complement (or be enhanced by) the other imaging modality.

An aspect of some embodiments of the invention relates to UVUS correction and/or reconstruction based on a 3D data set, for example, a CT data set. Optionally, the 3D data set is used as a scaffold for aligning, segmenting and/or correcting IVUS data. In an exemplary embodiment of the invention, slice-images acquired by IVUS are located in space, for example using a position sensor or using the image matching method described above and then matched to slices of the 3D data set. Optionally, the morphology of the 3D data set is used to identify borders in the IVUS image. Optionally, the IVUS image is re-aligned to match the orientation of the 3D data set. Optionally, the alignment of the IVUS is assumed to match a vessel centerline determined in the 3D data set. Optionally, data from the 3D data set is used to interpolate data missing in the IVUS set, for example, due to missing images or due to data not appearing on images.

There is thus provided in accordance with an exemplary embodiment of the invention, a method of estimating a position of a foreign object in the body, comprising:
(a) providing a 3D data set of at least one blood vessel;
(b) acquiring at least one 2D projection image of the vessel including the object;
(c) registering the 2D projection image of the vessel to the 3D data set; and
(d) using the registration to estimate a 3D position of said object restricted to be in a blood vessel, according to said 3D data set.

Optionally, said object comprises a catheter.

In an exemplary embodiment of the invention, said using comprises estimating a current position of said object based on an initial position of said object and an estimate of movement of said object along said vessel, using a measure of said vessel in said 3D data set.

In an exemplary embodiment of the invention, acquiring comprises acquiring an image with contrast material.

In an exemplary embodiment of the invention, further images are acquired without contrast material and for the purpose of detecting said object.

In an exemplary embodiment of the invention, the method comprises reconstructing a 3D IVUS data set using said position and IVUS data acquired by an IVUS head at said position for a plurality of IVUS head positions. Optionally, an orientation of said IVUS head is determined using a centerline of said blood vessel extracted from said 3D data set. Alternatively or additionally, reconstructing a 3D IVUS data set comprises arranging a plurality of IVUS images using said 3D data set as a scaffold.

In an exemplary embodiment of the invention, registering comprises segmenting said 3D data set to extract a vascular tree. Optionally, said projection image comprises a projected vascular tree and wherein registering comprises matching said vascular tree with said projected vascular tree.

In an exemplary embodiment of the invention, said 3D data set comprises a CT data set.

In an exemplary embodiment of the invention, acquiring comprises only acquiring a single projection image at a time.

In an exemplary embodiment of the invention, the method comprises enhancing said 3D data set with said 2D projection image. Optionally, enhancing comprises enhancing multiple projection images. Alternatively or additionally, enhancing comprises adding angiography data from said projection image to said 3D data set.

In an exemplary embodiment of the invention, the method comprises enhancing said 2D projection image with information from said 3D data set. Optionally, enhancing comprises indicating vulnerable plaque. Optionally, said vulnerable plaque comprises soft plaque.

In an exemplary embodiment of the invention, said image is an image of the heart. Optionally, acquiring comprises acquiring a heart-phase gated image. Optionally, using comprises using a gated image for a first position estimation and not limiting said using to a same phase of the heart for at least one subsequent image. Alternatively or additionally, the method comprises comparing images between phases to determine an amount of motion of said object relative to said vessel, caused by a heart phase.

In an exemplary embodiment of the invention, registering comprises estimating an initial relative orientation of said projection using a sensor on an imager used to acquire said projection image.

There is also provided in accordance with an exemplary embodiment of the invention, a method of image enhancement, comprising:
(a) providing a 3D data set of a patient including information not readily discernable in an x-ray angiography image;
(b) providing an x-ray angiography image of the patient; and
(c) generating an enhanced 2D image including at least an indication of some of said information.

Optionally, the method comprises identifying at least one structural feature in said 3D data set and using said feature for said enhancing. Optionally, said identifying comprises segmenting said 3D data set.

In an exemplary embodiment of the invention, said 3D data set comprises a CT data set.

In an exemplary embodiment of the invention, said indication comprises an indication of vulnerable plaque.

In an exemplary embodiment of the invention, said indication comprises an indication of outer remodeling.

In an exemplary embodiment of the invention, said 3D data set has a resolution lower than that of said 2D image.

In an exemplary embodiment of the invention, the method comprises enhancing said 2D image with information from a data set of another modality.

In an exemplary embodiment of the invention, generating comprises registering said image with said 3D data set.

There is also provided in accordance with an exemplary embodiment of the invention, a method of collecting IVUS data into a 3D data set, comprising:
(a) providing a 3D data set;
(b) providing a plurality of IVUS images;
(c) collecting said IVUS images into a 3D data set by at least one of:
   (i) arranging said IVUS images in space using said 3D data set as a scaffold;
   (ii) segmenting said IVUS images according to said 3D data set; and
   (iii) correcting said IVUS images according to said 3D data set.

Optionally, arranging comprises determining an orientation of at least one image by matching said image to said data set.

Optionally, arranging comprises aligning an axis of said IVUS with a centerline of a vessel determined from said 3D data set.

Optionally, segmenting comprises segmenting said 3D data set and segmenting at least one IVUS image according to said 3D data set segmentation. Optionally, segmenting at least one IVUS image comprises identifying an IVUS value as belonging to a tissue type based on a segmentation of the 3D data set.

In an exemplary embodiment of the invention, correcting comprises correcting for an effect of a shadow.

In an exemplary embodiment of the invention, the method comprises adding angiographic information from a projection image to said collected 3D data set.

In an exemplary embodiment of the invention, said 3D data set is of a lower resolution than said IVUS images.

In an exemplary embodiment of the invention, said collected 3D data set includes information extracted from said 3D data set. Optionally, said information comprises at least one surface.

In an exemplary embodiment of the invention, said information comprises at least one tissue identification.

In an exemplary embodiment of the invention, said information comprises at least bend or bifurcation location

In an exemplary embodiment of the invention, collecting comprises at least two of (i)-(iii).

In an exemplary embodiment of the invention, collecting comprises all three of (i)-(iii).

In an exemplary embodiment of the invention, the method comprises cleaning up said collected 3D data set. Optionally, cleaning up comprises removing tissue indications belonging to a vessel other than a vessel of interest.

There is also provided in accordance with an exemplary embodiment of the invention, apparatus comprising:
(a) a data input for x-ray projection data from an x-ray detector;
(b) a data input for IVUS data from an IVUS imager;
(c) a display; and
(d) a controller adapted and configured to generate a display using said IVUS data, based on a single projection from said detector.

In an exemplary embodiment of the invention, the apparatus comprises a data input for a 3D data set and wherein said controller is adapted to use said data set for generating said display.

In an exemplary embodiment of the invention, said controller is adapted to use multiple projection images.

In an exemplary embodiment of the invention, said apparatus comprises a C-arm imager.

### BRIEF DESCRIPTION OF THE FIGURES

Exemplary non-limiting embodiments of the invention will be described with reference to the following description of embodiments in conjunction with the figures. Identical structures, elements or parts which appear in more than one figure are generally labeled with a same or similar number in all the figures in which they appear, in which:

FIG. 1 shows a vascular tree, for example a coronary tree, including various indications such as a stent and narrowed regions;

FIG. 2 shows a vascular imaging system in accordance with an exemplary embodiment of the invention;

FIG. 3A is a flowchart of a method of using the vascular imaging system of FIG. 2, in accordance with an exemplary embodiment of the invention;

FIG. 3B is a flowchart of a method of processing of IVUS data, in accordance with an exemplary embodiment of the invention; and

FIG. 4 schematically shows an exemplary display for the vascular imaging system of FIG. 2.

### DETAILED DESCRIPTION OF EXEMPLARY EMBODIMENTS

Exemplary Vessel Tree

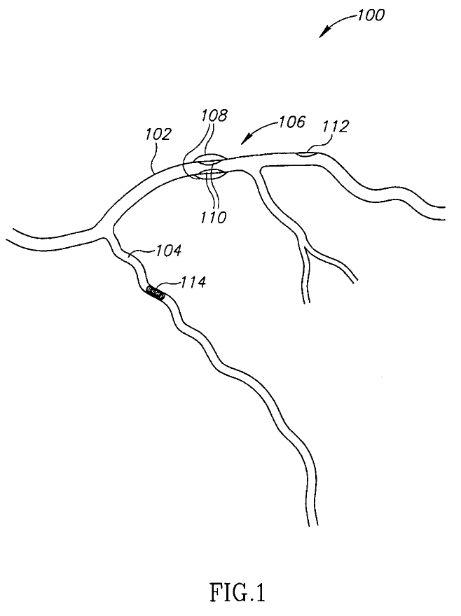

FIG. 1 shows a vascular tree 100, for example a coronary tree, including various indications such as a stent 114 and regions 106 and 112 narrowed by plaque. As shown the tree includes two main branches 102 and 104 and additional branches (un-numbered) as well.

Different diagnosis may be required for the different indications, for example, for stent 114, it may be desired to determine a degree of restenosis. For narrowing 108, it may be desirable to determine both a degree of outer remodeling 108 and a degree of lumen narrowing 110. In particular, a type and a dimension of narrowing 110 may be of interest. In an aneurysm (not shown), one or more of outer remodeling, plaque, effect of treatment and/or wall thickness may be of interest. For narrowing 112, which is not visible from the outer vessel diameter (and hence possibly less visible in some 3D imaging methods) the type of narrowing (e.g., soft plaque, calcification, shape, quality or area of attachment, vulnerability) may be of interest. In any blood vessel it may be interesting to know a degree of inflammation, noticeable, for example, as changes in wall thickness and/or in ultrasonic reflectivity.

While the invention focuses on coronary vessels, which generally have some unique problems, some methods of the invention may be applied to non vascular trees and/or non-coronary vessels, for example, the brain, peripherals vessels, abdominal aorta and kidney.

Exemplary System and Overview of Exemplary Method

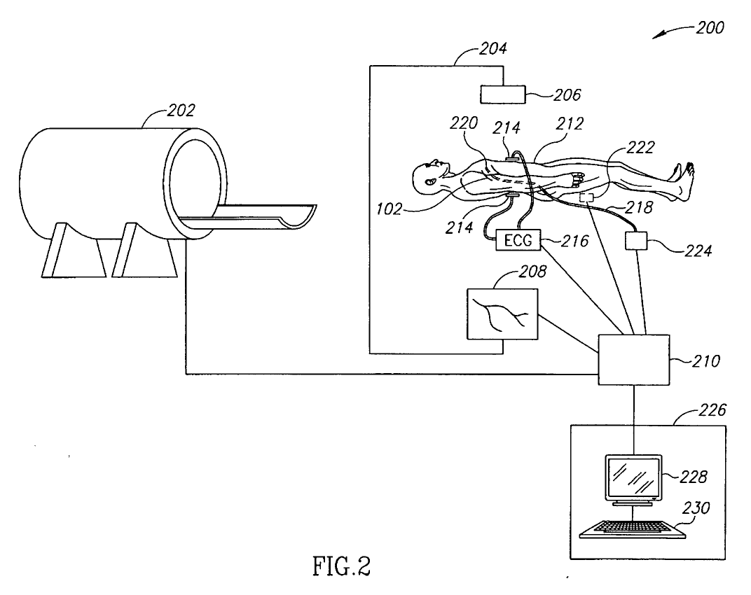

FIG. 2 schematically shows a vascular imaging system 200 imaging a patient 212 having vascular tree 100, in accordance with an exemplary embodiment of the invention. A 3D imager 202, for example a CT system is used to obtain a 3D image/data set of vascular tree 100 of patient 212. This image is sent to a controller 210. During an IVUS procedure, a catheter 218 with an IVUS head 220 is inserted into vascular tree 100 and imaged using an x-ray system, for example a C-arm single plane imager 204. A source 206 is provided at one side of the body and an image of tree 100, schematically shown as a reference 208, is acquired. This image is also sent to controller 210. As noted below, catheters other than IVUS catheter are used in some embodiments of the invention.

An optional ECG sensor 216, using a plurality of electrodes 214 is provided for estimating cardiac phase. Other methods may be used for estimating cardiac phase, as well, for example, image processing of image 208. Imager 204 is optionally gated responsive to a phase signal provided by ECG sensor 216 or another sensor.

In an IVUS mode, catheter 218 is inserted to a point for imaging and then pulled back. Imaging data from head 220 is optionally provided to controller 210 via a connector (optionally including control electronics) 224. A position sensor and/or pull back motor 222 is optionally used to provide controller 210 with an indication of movement of head 220.

In an exemplary embodiment of the invention, the CT data is used for one or more of registering to 2D projection data, generating catheter positions, combining with projection (e.g., angiography) or IVUS data and/or for assistance in reconstructing IVUS images. This and/or other data (as described below) is optionally processed and/or browsed on a station 226, optionally including a graphic display 228 and/or an input device such as a keyboard 230 and/or a mouse or stylus.

Exemplary Method

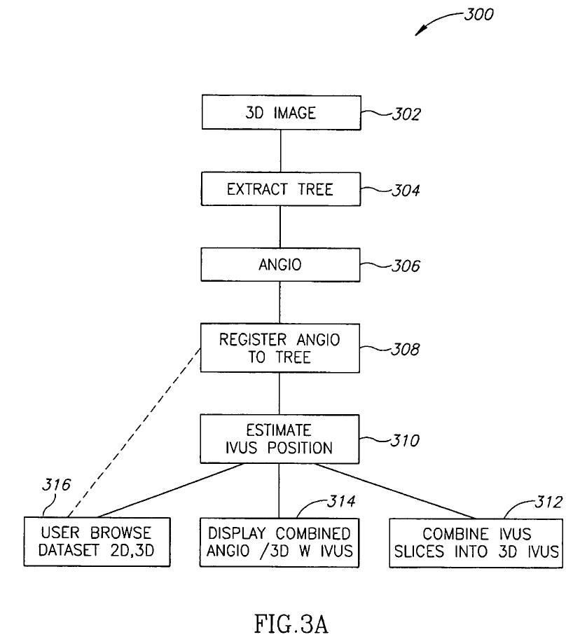

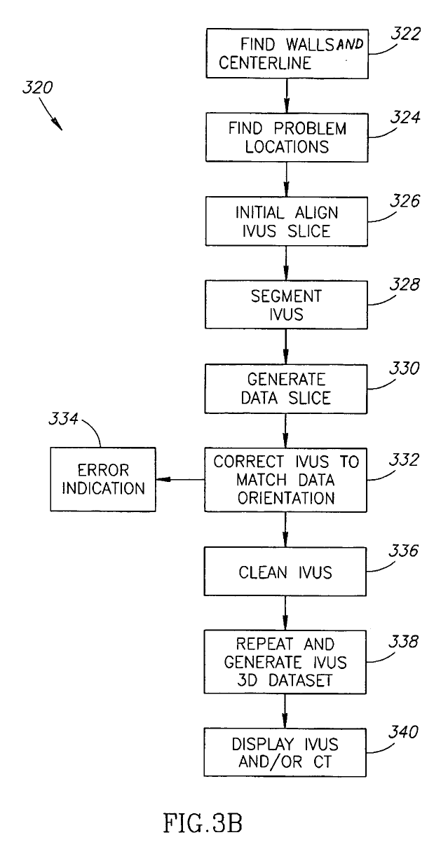

FIG. 3A is a flowchart 300 of a method of using vascular imaging system 200, in accordance with an exemplary embodiment of the invention.

3D Acquisition (302)

In an exemplary embodiment of the invention, a vascular tree is generated by first acquiring a 3D image of the area of interest (e.g., the heart). In an exemplary embodiment of the invention, the acquisition is by a CT system, for example, a multi-slice spiral ECG gated system. Optionally, an operator selects a phase of the heart in which an image is to be reconstructed. Alternatively, a multi-phase image, one 3D data set for each of several heart phases, is acquired. Optionally, the acquisition is carried out in synchronization with the injection of contrast material, using various methods as known in the art, for example, material injected using catheterization, or as a bolus.

In an alternative embodiment, the 3D image is acquired using other means, such as MRI, optical CT and/or ultrasound (e.g., external or trans-esophageal). Optionally, lower quality data, such as NM data is added on as a layer of data. Optionally, the imaging method is selected to complement the data which is acquired using IVUS and/or data acquired using x-ray angiography. In an exemplary embodiment of the invention, the imaging method is used to acquire data about one or more of calcifications, micro-calcifications, lumen diameter, vessel external diameter, inflammation and/or fatty plaque.

In the following description, there is a focus on the use of CT data, for example, MS CT data, however, this is provided for illustration only and in other embodiments of the invention, other imaging modalities can be used.

Tree Extraction (304)

Optionally, the 3D image data set is analyzed to identify blood vessels. Optionally, the blood vessels are extracted from the data as a vascular tree, for example the LAD may be extracted with its entire sub-vessels. Several methods are known in the art for such extraction, for example, “Knowledge-Based 3D Segmentation and Reconstruction of Coronary Arteries Using CT Images”, by Yan Yang, Allen Tannenbaum, Don Giddens, In Proceedings of the 26th Annual International Conference of the IEEE EMBS, pages 1664-1666, 2004. the disclosure of which is incorporated herein by reference.

Optionally, the tree is further processed to extract a centerline for the vessels in the tree, for example, using methods known in the art, such as Y. Kawata, N. Niki, and T. Kumazaki, “An approach for detecting blood vessel diseases from cone-beam ct image”, in IEEE Int. Conf. on Image Processing, pp. 500-503, 1995, and “Fast and Automatic Vessel Centerline Detection for MRA”, by K. Krissian C. F. Westin R. Kikinis, the disclosures of which is incorporated herein by reference.

In an exemplary embodiment of the invention, the desired quality of segmentation and centerline determination is for vessels with a healthy diameter of 2 mm, 1 mm or less. Optionally the precision of matching/registering (described below) of data to the CT data is within 3 mm, 2 mm, 1 mm or better. Optionally, the availability of multi-phase CT data is used to enhance the accuracy of matching/registration, using hyper-resolution and/or interpolation methods.

Optionally, a user indicates which parts of the tree are of interest. Optionally, the user makes this indication before or during the imaging. Alternatively, the indication can be after imaging. Optionally, the indication is carried out as part of the IVUS procedure (if any).

Optionally, the tree extraction is carried out ad hoc, when the tree is needed. In one example, portions of the tree are extracted when a determination of an extent of a target vessel is required. Actual presence of a blood vessel is optionally guessed based on Hounsfield unit values or on a different low quality vessel detection method. Alternatively or additionally, centerline determination and/or wall finding is provided ad hoc.

Optionally, tree extraction includes generating a model for other parts of the body, such as a heart (e.g., chambers, muscle, valves). Optionally, the image of the heart is used to assist a physician in orienting himself in the body, based on the relative location of the catheter and the heart. Disorientation sometimes occurs in which case a physician may confuse between two vessels that meet at a bifurcation. Optionally, additional extraction of data is performed, for example, extracting data from multiple phases (temporal data) and showing it spatially, for example, as a range of wall thicknesses and/or vessel positions.

Angiography (306)

Typically, IVUS is carried out during an angiography procedure or in conjunction with one. For example, catheter 218 (FIG. 2) is used to inject a contrast material at a suspected narrowing region in tree 100. Optionally, the angiography is carried out using the 3D imaging method above, for example, using CT angiography.

Even if angiography is not carried out, IVUS or other catheter based procedures are optionally carried out using x-ray imaging, for example, fluoroscopy, to monitor the motion of the catheter in the body.

In an exemplary embodiment of the invention, x-ray imaging uses an ECG-gated C-arm system (204), for example a Philips Allura Xper system.

Optionally, the position and/or orientation of imager 204 relative to patient 212 are determined, for example, manually, or using one or more sensors. Various position and/or orientation sensors are known in the art and may be used. In such systems, the registration process described below is optionally replaced by a geometry based registration process, or the position sensors may be used as an initial guess for more accurate image based registration.

Registration (308)

In an exemplary embodiment of the invention, image 208 acquired by imager 204 is registered to the 3D data set acquired by imager 202. Various methods known in the art can be used to register the image to the 3D data set, for example as described in the background section.

In an exemplary embodiment of the invention, the image acquired by imager 204 is gated to be at a same cardiac phase as the data set from imager 202. If the data set is multi-phase, one of the phases is optionally selected for gating. Alternatively, multi-phase x-ray images may be acquired. Optionally, the cardiac phase is determined by ECG. Alternatively, image characteristics may be used for such determination. For example, the diastole may be identified as a period with minimum movement of the heart walls and/or vessels and a duration of the period above a certain threshold. Optionally, the phase is detected by finding a phase value for which the error in matching to the data set is minimal.

In an exemplary embodiment of the invention, the following method is used to find a transformation between the 2D imager 204 coordinates and 3D imager 202 coordinates.

In general a C-Arm imager (204) can be approximately modeled as a perspective pinhole camera (without reversing). The process of transforming a point from a 3D world coordinate to a 2D C-Arm image coordinate can be presented as four steps, some of which may be omitted.
Step 1: Rigid Transformation from the world (or CT) coordinate system to the C-Arm coordinate system.
Step 2: Transformation from the 3D C-Arm coordinate to an undistorted image coordinate using perspective projection with pinhole camera geometry.
Step 3: Spatial distortion of image coordinates (e.g., based on collimator and/or detector geometry).
Step 4: Physical image coordinate to computer image coordinate transformation.

The parameters used in these four steps can be categorized into two classes:
1. Intrinsic Parameters:
   The effective focal length, or image plane to projective center distance, f; the image center, (cx, cy); the pixel scaling, (px, py); and the distortion map, M(x,y).
2. Extrinsic Parameters:
   The Euler angles yaw α, pitch β, and tilt γ for a Rotation matrix R and the three components (Tx, Ty, Tz) for a translation vector T.

An Intrinsic Transformation can be calculated from C-Arm parameters data. In some cases, the 2D projected image obtained is already after distortion map correction by the C-arm imager.

Regarding the rigid transformation process. Optionally, the transformation is formulated as a transformation of point X in a source image (i.e. a MSCT 3D vessel location) into the coordinate space Y of a target image (i.e. the X-Ray 2D projected vessel). A rigid transformation occurs as a rotation matrix multiplication plus a translation
Y=XR+T (1)
Where R is an Euler rotation matrix parameterized by α, β, γ as rotations about the z, y, and x-axes respectively and where T is a three dimensional offset vector.

In an exemplary embodiment of the invention, a registration metric is used, to quantify how well a rotation matrix and an offset vector align a vascular model from one vascular image with another vascular image. In an exemplary embodiment of the invention, the metric is based on an assumption that the vessel centerlines are scaled intensity ridges in vascular images; therefore, when a vascular model is aligned with a vascular image, the centerline points in the model should map to bright points in the image. A cost function is defined for this correlation and optimization is performed on this function to achieve a good registration.

Optionally, other feature extraction and/or matching methods are used. Various image (including 2D intensity image) matching methods are known and may be used.

In an exemplary embodiment of the invention, the optimization determination is enhanced by using the orientation of imager 204 (angle from RAO and angle from Caudal) as a coarse projection direction.

Once the transformation is determined, data can be combined between the two data sets, for example, data can be blended or one data can be used as a scaffolding for other data. In an exemplary embodiment of the invention, the higher resolution IVUS data is used to enhance the CT data, for example, using the CT data to show surfaces (e.g., segmentation) and using IVUS data to show properties within the segments. In another example, an angiography image is enhanced by marking soft plaque or other vascular structural features (e.g., extracted from the 3D data set) on it. Additional details are provided below.

IVUS Position Estimation (310)

In an exemplary embodiment of the invention, the registration of the 2D x-ray image to the 3D data set is used for estimating the position of IVUS head 220.

In an exemplary embodiment of the invention, estimation has two parts:
a) estimating an initial position of head 220 (or another part of the catheter); and
b) estimating a current position of head 220.

In an exemplary embodiment of the invention, the initial position is estimated by acquiring an x-ray image and determining the above described registration. The location of IVUS head 220 is optionally inputted manually. Alternatively, the position is optionally detected automatically, for example by automatic detection of a radio-opaque marker built into head 220. As head 220 is known to be in a blood vessel, a search is made for a blood vessel that, when projected onto the 2D image encloses head 220. Optionally, if there is more than one possibility, a user may select the vessel on the 3D image, for example, being given the option to select among multiple possibilities. Optionally, if there is overlap, the patient and/or imager 204 are moved, to reduce ambiguity. Optionally, catheter 118 also includes one or more radio-opaque markers, which are used to further limit the possible vessels to be considered (as head 220 and catheter 118 must lie in a same vessel in a normal procedure). Optionally, the vessel is identified by head 220 being moved and then generating multiple head position transformations. The constraint on head position can now be requiring that a same vessel is found which matches all the head transformations. In some cases the vessel is perpendicular (or nearly is) to the plane of x-ray imaging, so there are many positions along the vessel that could match. Optionally, any ambiguity in catheter position is shown on the display. Optionally, a bi-plane image is acquired or imager 204 is rotated to reduce the ambiguity. Optionally, an audio or visual signal or message is provided to an operator to rotate the imager.

Optionally, a current position is determined by further x-ray acquisition. In an exemplary embodiment of the invention, a tip detection method is used in which low radiation doses are used and no contrast material is required, for example using a catheter with a radio-opaque marker.

In some cases, however, it is desired to further minimize exposure to radiation and/or contrast material. In an exemplary embodiment of the invention, a current head position is estimated based on the initial position and a length of pull-back of the IVUS head during imaging. Typically IVUS is used by first reaching a position and then pulling back the IVUS head. Optionally, the IVUS head is moved forward. Optionally, a motor and/or axial movement sensor (222) are used to determine catheter head motion. Various types of sensors may be used, for example, a linear encoder in which a sensor mounted on a vascular port reads a marking from catheter 218. Optionally, a fixed speed motor or a stepper motor are used, in which the command to the motor may be used as an indication of position.

Optionally, the axial position along the vessel is estimated using a true length along the vessel centerline in the 3D data set, rather than a projected distance in an angiography image. The angiography image may be updated by annotating it to include the current position of head 220. Additional information, such as vessel widths measured in the 3D data set, may be added as well. The updated image may be shown, for example, on display 228 or being fed back to a display of imager 204. Alternatively, a 3D presentation is used, as described below, for example. A 3D display, for example of a type known in the art, is optionally used.

In an exemplary embodiment of the invention, the orientation of IVUS head 220 (and thus the UVUS slice orientation, which is typically perpendicular) is estimated by assuming the head lies along the vessel centerline at which the head is located. Optionally, this estimate is verified using the projection of the head on the 2D image.

In an exemplary embodiment of the invention, IVUS head orientation is determined or verified by aligning the IVUS image with a matching spatial section of the 3D data set. Changes in obliqueness of the vessel may be used to indicate an orientation problem. Changes in vessel diameter may indicate an axial positioning problem. The position may be adjusted until an error is minimal. Optionally, the comparison takes into account parts of the data set when an increased error may be expected, for example, at bends. Optionally, if the error is above a threshold, the user is notified. Optionally, catheter position is constrained by one or more real-world values, such as vessel lumen (e.g., as measured by CT), catheter flexibility and/or catheter length inside the body.

In an exemplary embodiment of the invention, position estimation is carried out at other phases of the heart, for example, using the above estimation method. Optionally, once the initial position of the catheter is known, its position can be estimated at any phase, based on the axial length of the catheter in the body. Optionally, the same data set is used for all phases. Alternatively, a different data set is used for different phases. Optionally, data acquired at some phases is more reliable than at other phases. For example, the catheter might bend only at some phases. Optionally, this is detected by comparing the IVUS images and/or projection images between phases to detect an existence and/or degree of such changes.

Optionally, the IVUS head is rotated around its axis, for example, while stationary or while being moved back. Such rotation may be useful for a directional system. Optionally, the rotational direction of IVUS head 220 is reported by at least one rotation sensor in head 220 and/or by comparing the image to a structure identified in the 3D data set.

3D IVUS Generation (312)

In an exemplary embodiment of the invention, IVUS head 220 generates trans-axial images (slices) of the vessel in which it is located. These slices may be collected using various methods and formed into a 3D image data set. One method of collection comprises aligning the images based on the head position and/or orientation and interpolating values for data set elements that lie between the slices based on the distance between images (e.g., based on relative motion) and pixels in those images. Various interpolation methods may be used, for example, linear, bi-linear and spline. Optionally, 3D data set data is used for interpolation (alone or with a weight) where IVUS data is missing or far away. Optionally, an orientation or tilt sensor is provided on head 220 to help determine its orientation. Optionally, a bi-plane x-ray imaging method is used to obtain orientation data.

In an exemplary embodiment of the invention, the IVUS data is realigned, segmented and/or corrected using the 3D data set. In an exemplary embodiment of the invention, the data set, for example a CT data set is used as a scaffold that provides a structure to which the IVUS data is expected to conform. FIG. 3B is a flowchart 320 showing an exemplary method of processing of IVUS data according to a 3D data set.

At 322, the 3D data set is processed, for example to determine one or more of vessel wall and vessel centerlines. If the vascular tree is not complete, it may be completed. Many methods of determining these features are known.

At 324, problem locations in the 3D data set are optionally identified, for example, bifurcations (e.g., based on geometry), curves (e.g., based on geometry), plaque (e.g., based on CT number). Optionally, the problem areas are areas that have health problems. Alternatively or additionally, the problem areas are areas where matching to an IVUS image may be problematic.

At 326, an initial positioning of an IVUS slice is optionally performed. O the IVUS slice is arranged to be centered on and perpendicular to the vessel centerline. Alternatively or additionally, the slice is positioned so its lumen corresponds to the vessel lumen. Other ways of estimating slice orientation are described above. The axial position is optionally by position determination (e.g., based on a position sensor or x-ray imaging) or based on an estimate, such as using the estimation method described above.

At 328, the IVUS slice is optionally segmented. In an exemplary embodiment of the invention, a method such as described in Sonka, M., McKay, C. R., and von Birgelen, C: “Computer Analysis of Intravascular Ultrasound Images” in C. T. Leondes, editor, Medical Imaging Techniques and Applications, Gordon and Breach, pp. 183-226, 1997, the disclosure of which is incorporated herein by reference, is used.

At 330, a CT (for example) slice corresponding to the IVUS slice is generated from the 3D data set.

At 332, the IVUS position and/or orientation are corrected according to its correspondence to the CT slice and/or vessel centerline. In an exemplary embodiment of the invention, the CT and IVUS slices are matched according to the CT segmentation (322) and the IVUS segmentation (328). Optionally, even if the IVUS is not segmented, there is expected to be a correlation between the border on the IVUS image slice and on the CT segmentation. Optionally, obliqueness of the IVUS relative to the CT is used to estimate a correction for orientation of the IVUS.

Optionally, 328 and 330 are replaced by a different process in which a maximum correlation between the IVUS data and the 3D data set data is found under a limited range of rotations and/or stretching of the IVUS data.

At 334, an error message or mismatch indication is optionally provided to a user. Optionally, the indication is as a message or as a marking on an image display.

At 336, the IVUS data is optionally cleaned. In one example, image areas which appear to be outer-remodeling but are actually caused by a nearby bifurcation, are identified in the CT segmentation and removed (or marked) in the IVUS data set. Other elements which may be cleaned off include shadow areas. Optionally, an IVUS image is corrected, for example, amplitude enhanced, at shadow areas, to reflect a known tissue property of tissue in the shadow area. Amplitude enhancement according to CT Houndsfield unit values may be used to correct for shadow artifacts, so that all IVUS areas known to be of a same tissue type will have similar average amplitude. This may be performed at a later time as well, for example, on the data set as a whole. Other exemplary clean-up operations include spatial distortion, to match the CT data set.

At 338, acts 326-336 are optionally repeated until a complete 3D data set for the IVUS data is created. Missing data is optionally provided by interpolation with IVUS and/or CT data. Additional data, for example provided by the projection images may be added as well.

At 340, the IVUS and/or CT data are shown. Optionally, the structure (e.g., borders) are based on CT data while the actual measurements are IVUS measurements. Optionally, data outside of a segment is cleaned up, for example, noise may be removed in a lumen of a vessel or outside a vessel wall. Optionally, a divergence between IVUS and CT data is allowed, for example to within one, two or more resolution units of the CT data. Within this range, IVUS data is allowed to not match the CT segmentation. In an exemplary embodiment of the invention, the relative resolutions of CT and IVUS is, 1:3, 1:5 or better, for example, the CT being 0.5 mm in voxel size and the IVUS being 0.1 mm (in some directions). Optionally, lower resolution CT images are acquired if IVUS data will be (or is) available, this may reduce radiation doses and/or contrast material by, for example, 30%, 60%, 75% or intermediate or greater reduction.

Display Combined Data (314)

In an exemplary embodiment of the invention, the data from the various modalities is combined together. The combined data can be shown, for example, as a 3D data set, as a 2D projection (e.g., in the format of an angiogram) or in other ways, for example, as a movie of traveling along the vessel, as a series of slices along the vessel and/or as a set of two or more synchronized views where marking on one view or moving on one view generates a corresponding movement/marking on the other view, for example, a virtual IVUS view and a 3D volume view.

Optionally, the combined data is analyzed, for example, to extract various statistical information, such as average vessel inner and outer diameter, average calcification count, area of attachment (absolute and/or ratio to amount of plaque), area of contact of plaque with blood, calcium and/or plaque scoring (which is optionally used for diagnosis and/or suggested treatment) and/or temporal information, such as diameter change as a function of phase.

In an exemplary embodiment of the invention, the combination of data is shown in a spatial manner. In this example, 3D data set is projected onto the angiography image and supplements it. A set of one or more combination rules may stipulate that when there is data from two sources, one of the sources is selected or a certain combination is made. In some cases, the data from one source complements the other source in a functional manner. For example, IVUS data and x-ray data of a same narrowing, reflect different characteristics of a tissue. In an exemplary embodiment of the invention, enhancement data is shown as color layers and/or highlighting of a black and white or color image.

Optionally, the process of data combination is used to quantify the quality of image matching. For example, if an IVUS image shows tissue, where a CT image shows a bolus of contrast material, an error is flagged. Optionally, a user settable threshold is used to decide on error conditions.

Optionally, the data is combined to form multi-phase data, for example,

In an exemplary embodiment of the invention, the data from different modalities are synchronized using ECG. Optionally, breathing synchronization is used as well or instead.

In an exemplary embodiment of the invention, the 3D data set is analyzed to determine if for a particular position movement artifacts are a problem and/or are heart-phase related. In an exemplary embodiment of the invention, once the first IVUS point is correctly determined, a position even between phases can be estimated based on movement of the catheter. Optionally, the methods described in FIG. 3B are used to correct for motion artifacts. Optionally, some motion artifacts are desirable, for example, vessel change that indicates vessel flexibility and/or arrival of a pulse wave. Optionally, the diameter of the vessel is adjusted when combining data from different modalities with different phase values. Optionally, the CT data is distorted (e.g., vessel diameter changed) to match the phase-dependent data provided by the IVUS. Optionally, the distortion takes the stiffness of plaque into account (e.g., hard plaque not being distorted.

Browsing in Data (316)

Once the angiography image (208) is registered to the 3D data set (308), the data (e.g., 3D data set, projection image, IVUS and/or a combination data set thereof) can be browsed in various ways. Optionally, the x-ray image is a non-angiography image.

In an exemplary embodiment of the invention, the 2D image is browsed, optionally, after the 2D image is enhanced with information from the 3D image.

In an exemplary embodiment of the invention, the 3D data set is browsed, optionally, after the data set is enhanced using information from the 2D image. Optionally, when it is not clear to which vessel on the 3D image information from the 2D image is mapped, the data is marked, for example, by highlighting with color on a 3D view of the vessel and/or the heart.

In an exemplary embodiment of the invention, the data is browsed by generating a virtual UVUS-like image from the 2D and/or 3D data set. In an exemplary embodiment of the invention, data from a 2D projection image is copied to the 3D data set by evenly distributing the data from the projection image at the various orientations around a blood vessel that correspond to the location of data on the projected image. If multiple direction of projection images are provided, the distribution is optionally constrained to match their distribution. Optionally, the distribution is made to match a structural indication, such as provided by a CT data set.

In an exemplary embodiment of the invention, the following visualization method is used. The projection is shown overlaid on the 3D data set and then the 3D data set is manipulated, for example, rotated by the user. The projection then disappears, however, a visual memory of the projection can help a user match the projection to the 3D data set.

In an exemplary embodiment of the invention, segmentation of the data set to show heart geometry is used to assist a user in identifying blood vessels based on their position on the heart.

In an exemplary embodiment of the invention, the processed data set also includes data from the IVUS image. Optionally, a smooth change is provided when viewing images, when the location of imaging changes between an IVUS area and a non-IVUS area.

In an exemplary embodiment of the invention, the combined data is printed, for example, as an enhanced angiograph (e.g., showing outer modeling, plaque type).

In an exemplary embodiment of the invention, the combined data is used to guide treatments and decisions, for example, PCTA, stent positioning and/or clot and narrowing dissolution and/or removal methods.

In an exemplary embodiment of the invention, a virtual IVUS image is generated. In this image, CT data is modified and shown as if it were generated by IVUS. In an exemplary embodiment of the invention, the CT data set is processed by selecting slices corresponding to a beam section (e.g., possibly conical) of IVUS and optionally degraded in a radial direction (e.g., degradation in contrast and/or noise). The blood with contrast material is identified and ignored in the image, such that only values above and below a range of values of the blood are shown. For example, soft plaque has values below blood and hard plaque can have values above blood. Optionally, dead zones of the IVUS are shown as blank.

Exemplary Display and Analysis

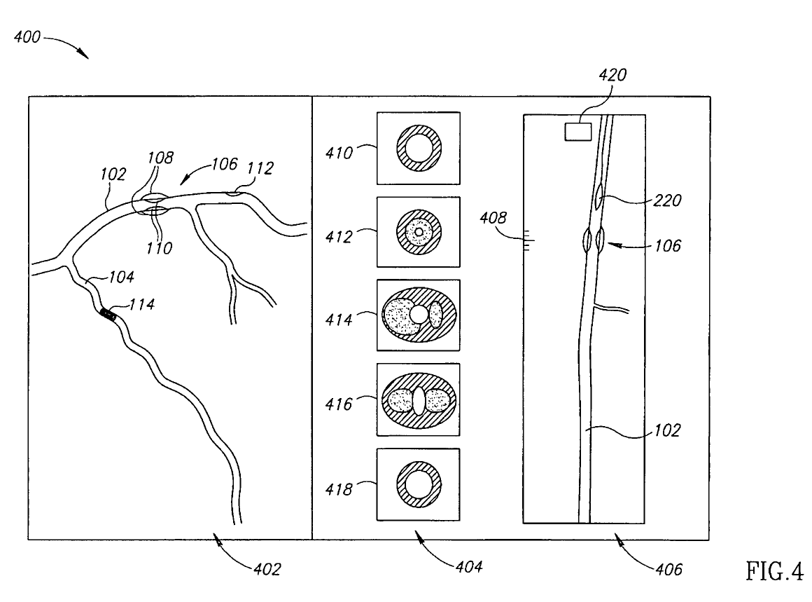

FIG. 4 shows an exemplary display 400 for vascular imaging system 200. In display 400, three image areas are used, a tree display area 402, which shows tree 100, optionally with additional image data, such as the heart or other organs; an IVUS slice display area 404 and a vessel plan view area 406 which shows a vessel of interest laid out. Optionally, one or more of the areas can be changed, for example, area 402 or 404 being used to show an IVUS video stream. In an exemplary embodiment of the invention, the positions in the image areas are correlated, so that if there is a user marker on one area, its corresponding indication is shown on the others.

In an exemplary embodiment of the invention, area 402 is used for general overview and for selection of regions of interest, for example selecting a target vessel section to be viewed in areas 404 and 406. Additional data, for example, vessel centerlines and heart parameters may be shown in area 402 as well, or in other areas. Alternatively or additionally, function data may be shown, for example, wall thickness, perfusion, and wall motion. Optionally, nuclear medicine imaging data or echocardiogram data is overlaid on the image. Optionally, the data is overlaid as layers. Alternatively, the data is merged into the image, for example, using color layers. Optionally, the heart and/or vessels are shown as a background, which is optionally muted in color and/or partially transparent.

In an exemplary embodiment of the invention, a user can measure a length of a vessel, by marking two or more points on the vessel (optionally, in any of the display areas). In an exemplary embodiment of the invention, the length is measured by determining the starting and ending position of head 220 in the vessel and measuring the amount of pullback of catheter 118. In the figure, a real or artificial image of head 220 in the target vessel is shown. If needed, the image of head 220 or catheter 218 may be removed.

In an exemplary embodiment of the invention, length is measured on the centerline, for example as determined from the 3D data set. Other measurements may be extracted from the 3D data as well, for example, vessel volume, average plaque density, size, inflammation values (e.g, from MRI) and/or shape. Such measurements can be shown in various ways, for example, in a value section 420, or as text callouts.

In an exemplary embodiment of the invention, the display shows operational information, such as precision of matching and/or spatial resolution.

In the example shown, a plurality of marks 408 indicate where IVUS slices are generated. These slices are shown as slices 410-418 and schematically illustrate various narrowing and outer modeling of a blood vessel by plaque.

Referring back to area 406, various items of interest can be seen, for example, narrowing 106, IVUS head 220 and side branches. Optionally, this area is used to view an implanted stent. Optionally, angiography data and/or IVUS data are used to supplement CT or MRI 3D data if such data is distorted by the stent itself.

In an exemplary embodiment of the invention, one or more of the displayed images are processed and/or corrected. In one example, the exact location of a curve or bifurcation is marked in area 406. In an exemplary embodiment of the invention, the ability to mark the bifurcation relative to the IVUS data allows a better appreciation of the disorder. Optionally, a plan view of the vessel (e.g., the vessel is split and laid flat) is shown instead of a trans-axial slice of the vessel. In a plan view, colors and/or textures may be used to indicate plaque type, narrowing and/or other problems. Vessel outer remodeling may be shown, for example, by the plan view being wider at bulging points.

In an exemplary embodiment of the invention, the vessel image is corrected for the effect of the catheter on vessel geometry, for example, if catheter 218 or head 220 cause local diameter and/or curvature changes, these effects are corrected for by using the 3D data set as a reference. Optionally, as noted above, IVUS and angiographic data may be used to enhance a CT data set.

In an exemplary embodiment of the invention, display 400 is used during a catheterization procedure, for example to show plaque before and/or after being treated and/or show old and/or new stent locations. Optionally, changes before or during treatment are shown, for example, as overlays or by highlighting. Optionally, clicking on an area allows a user to view the change.

Optionally, the 3D data set shown in area 402 is enhanced by showing the vessels as at least partially transparent and showing internal information as obtained by angiography or IVUS.

In an exemplary embodiment of the invention, IVUS data is used to determine how to redistribute angiography information in a 3D space. For example, if the IVUS shows more plaque on one size of a vessel than on another side thereof, the angiography density data is redistributed accordingly.

In an exemplary embodiment of the invention, as a procedure progresses, projection data is used to incrementally build a 3D data set. Optionally, the CT is used as a scaffold to indicate where data from the projection images should be associated with. Optionally, the data in the 3D data set is reprojected using a weighted version of the projection data. Alternatively, other image reconstruction methods are used.

In an exemplary embodiment of the invention, the 3D data set is shown as a surface image, for example, a single (selected) vessel being shown, with IVUS slices overlaid on the vessel and the vessel itself semi-transparent. Surfaces, as defined by CT segmentation and/or IVUS segmentation are optionally displayed as smooth surfaces rather than pixels.

Variations

While the above has focused on IVUS based systems, some of the method and apparatus described above, can be carried out on other systems, without IVUS, for example, CT and angiography systems.

In an exemplary embodiment of the invention, the matching of an angiographic image to a 3D data set is used to locate a catheter and/or its tip that are visible on the angiographic (or other single plane or bi-plane projection image). Optionally, the method described in Shirley A. M. Baert, Max A. Viergever, Wiro J. Niessen: “Guide Wire Tracking During Endovascular Interventions”, IEEE Trans. Med. Imaging 22(8): 965-972 (2003), the disclosure of which is incorporated herein by reference, is used. Optionally, the catheter position is shown overlaid on the 3D data set. In an exemplary embodiment of the invention, the catheter/tip position is used also for advancing of a catheter, optionally without contrast material, for example, using the vascular tree and either low-level radiation imaging or an estimate of the length/movement of the catheter in the body. Optionally, body movement is corrected for, for example, by automatically aligning consecutive images.

In an exemplary embodiment of the invention, the angiographic or other, x-ray image, is combined with the 3D data set and shown as a 2D or 3D data set.

In an exemplary embodiment of the invention, the combined information is used during a catheterization procedure as a help for making decisions. For example, once a plaque type and size are identified, it can be decided if to use a stent, the type of stent and/or to use PCTA.

In the above description of system 200, a certain distribution of processing was described. However, the data can be acquired in various ways and processed in various ways, including more distributed and more centralized. For example, a same system can be used for all of the processing and optionally for sending the data to a user. Such processing can be in real-time or offline. In another example, some or all of the processing is performed off-line, after the fact by a user station that receives the different data and images and then reconstructs a display and/or makes measurements based on this processing. X-ray, CT and/or IVUS data sets may be stored locally and/or remotely, for example accessible by network.

In another example, the CT data set can be used to determine what viewing angle is best for projection imaging (e.g., for viewing and/or for image registration) and instructions to use that direction are issued.

In an exemplary embodiment of the invention, the methods described herein are used to identify a position inside a heart chamber. Optionally, the position is correlated with electrical and/or other measurements taken by the catheter at the position, for example using methods known in the heart. In an exemplary embodiment of the invention, the geometry of the chamber is known from the CT segmentation. Optionally, in case of ambiguity (which can be expected for a significant portion of the cases) the user is requested to rotate the imager or a automatic rotation is carried out. Optionally, a bi-plane imager is used, which includes sufficient data for position reconstruction.

While the above has focused on methods, in an exemplary embodiment of the invention, a computer is programmed and/or circuitry built to carry out the method. Optionally, an existing system is upgraded (e.g., by software and/or adding data inputs and/or outputs and/or control ability). In some cases additional memory and/or processing ability may be desirable. In an exemplary embodiment of the invention, the software of a C-arm system is updated to perform the methods described above. Optionally, the software (and/or hardware) provided includes one or more of the following modules, it being clear that in some implementations of the systems, various modules are combined and/or split up other than described herein:
(a) an input module adapted to receive one or more of a 3D data set (e.g., CT), IVUS data and x-ray imager data;
(b) segmentation software for segmenting, vessel tree extraction and/or centerline determination for CT and/or IVUS;
(c) registration software for registering CT data to projection data, which may include an input of C-arm orientation and/or position;
(d) data merging software for combining and/or enhancing a data set of one modality using another modality;
(e) viewing software for showing data sets, optionally including the ability to show a 3D IVUS data set and/or virtual IVUS
(f) locating software for locating a catheter and/or catheter tip in a 2D and/or a 3D image;
(g) software and/or hardware for measuring or estimating (e.g., based on motor speed) a degree of insertion and/or movement of a catheter in the body;
(h) software for reconstructing a 3D data set form IVUS images, optionally using a 3D data set as a scaffold; and
(i) software for reconstructing or enhancing a 3D data set from/using multiple angiography projection images.

It will be appreciated that the above described methods of image processing may be varied in many ways, including, changing the order of steps, which steps are performed more often and which less often, the arrangement of the imager(s), the type and order of images acquired and/or the particular pathologies looked for. Further, the location of various elements may be switched, without exceeding the sprit of the disclosure, for example, the location of the image memory. In addition, a multiplicity of various features, both of method and of devices have been described. It should be appreciated that different features may be combined in different ways. In particular, not all the features shown above in a particular embodiment are necessary in every similar exemplary embodiment of the invention. Further, combinations of the above features are also considered to be within the scope of some exemplary embodiments of the invention. In addition, some of the features of the invention described herein may be adapted for use with prior art devices, in accordance with other exemplary embodiments of the invention. Further, various means for carrying out the above described functions are included in the scope of the invention, for example, imaging means, processing means and/or image combining means. The particular geometric forms used to illustrate the invention should not be considered limiting the invention in its broadest aspect to only those forms, for example, where a C-arm is shown, in other embodiments a different type imager is used. Although some limitations are described only as method or apparatus limitations, the scope of the invention also includes apparatus programmed and/or designed to carry out the methods, for example using firmware or software programming and methods for electrifying the apparatus to have the apparatus's desired function.

Also within the scope of the invention are kits for single use or limited use, for example, a set comprising contrast material and code(s) for using the software. Section headers are provided only to assist in navigating the application and should not be construed as necessarily limiting the contents described in a certain section, to that section. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A method of estimating a position of a foreign object in the body, comprising:
(a) providing a 3D data set of at least one blood vessel;
(b) acquiring at least one 2D projection image of the vessel including the object;
(c) registering the 2D projection image of the vessel to the 3D data set; and
(d) using the registration to estimate a 3D position of said object restricted to be in a blood vessel, according to said 3D data set.

2. A method according to claim 1, wherein said object comprises a catheter.

3. A method according to claim 1, wherein said using comprises estimating a current position of said object based on an initial position of said object and an estimate of movement of said object along said vessel, using a measure of said vessel in said 3D data set.

4. A method according to claim 1, wherein acquiring comprises acquiring an image with contrast material.

5. A method according to claim 4, wherein further images are acquired without contrast material and for the purpose of detecting said object.

6. A method according to claim 2, comprising reconstructing a 3D IVUS data set using said position and IVUS data acquired by an IVUS head at said position for a plurality of IVUS head positions.

7. A method according to claim 6, wherein an orientation of said IVUS head is determined using a centerline of said blood vessel extracted from said 3D data set.

8. A method according to claim 6, wherein reconstructing a 3D IVUS data set comprises arranging a plurality of IVUS images using said 3D data set as a scaffold.

9. A method according to claim 2, wherein registering comprises segmenting said 3D data set to extract a vascular tree.

10. A method according to claim 9, wherein said projection image comprises a projected vascular tree and wherein registering comprises matching said vascular tree with said projected vascular tree.

11. A method according to claim 2, wherein said 3D data set comprises a CT data set.

12. A method according to claim 1, wherein acquiring comprises only acquiring a single projection image at a time.

13. A method according to claim 2, comprising enhancing said 3D data set with said 2D projection image.

14. A method according to claim 13, wherein enhancing comprises enhancing multiple projection images.

15. A method according to claim 13, wherein enhancing comprises adding angiography data from said projection image to said 3D data set.

16. A method according to claim 2, comprising enhancing said 2D projection image with information from said 3D data set.

17. A method according to claim 16, wherein enhancing comprises indicating vulnerable plaque.

18. A method according to claim 17, wherein said vulnerable plaque comprises soft plaque.

19. A method according to claim 2, wherein said image is an image of the heart.

20. A method according to claim 19, wherein acquiring comprises acquiring a heart-phase gated image.

21. A method according to claim 20, wherein using comprises using a gated image for a first position estimation and not limiting said using to a same phase of the heart for at least one subsequent image.

22. A method according to claim 20, comprising comparing images between phases to determine an amount of motion of said object relative to said vessel, caused by a heart phase.

23. A method according to claim 1, wherein registering comprises estimating an initial relative orientation of said projection using a sensor on an imager used to acquire said projection image.

24. A method of image enhancement, comprising:
(a) providing a 3D data set of a patient including information not readily discernable in an x-ray angiography image;
(b) providing an x-ray angiography image of the patient; and
(c) generating an enhanced 2D image including at least an indication of some of said information.

25. A method according to claim 24, comprising identifying at least one structural feature in said 3D data set and using said feature for said enhancing.

26. A method according to claim 25, wherein said identifying comprises segmenting said 3D data set.

27. A method according to claim 24, wherein said 3D data set comprises a CT data set.

28. A method according to claim 24, wherein said indication comprises an indication of vulnerable plaque.

29. A method according to claim 24, wherein said indication comprises an indication of outer remodeling.

30. A method according to claim 24, wherein said 3D data set has a resolution lower than that of said 2D image.

31. A method according to claim 24, comprising enhancing said 2D image with information from a data set of another modality.

32. A method according to claim 24, wherein generating comprises registering said image with said 3D data set.

33. A method of collecting IVUS data into a 3D data set, comprising:
(a) providing a 3D data set;
(b) providing a plurality of IVUS images;
(c) collecting said IVUS images into a 3D data set by at least one of:
(i) arranging said IVUS images in space using said 3D data set as a scaffold;
(ii) segmenting said IVUS images according to said 3D data set; and
(iii) correcting said IVUS images according to said 3D data set.

34. A method according to claim 33, wherein arranging comprises determining an orientation of at least one image by matching said image to said data set.

35. A method according to claim 33, wherein arranging comprises aligning an axis of said IVUS with a centerline of a vessel determined from said 3D data set.

36. A method according to claim 33, wherein segmenting comprises segmenting said 3D data set and segmenting at least one IVUS image according to said 3D data set segmentation.

37. A method according to claim 36, wherein segmenting at least one IVUS image comprises identifying an IVUS value as belonging to a tissue type based on a segmentation of the 3D data set.

38. A method according to claim 33, wherein correcting comprises correcting for an effect of a shadow.

39. A method according to claim 33, comprising adding angiographic information from a projection image to said collected 3D data set.

40. A method according to claim 33, wherein said 3D data set is of a lower resolution than said IVUS images.

41. A method according to claim 33, wherein said collected 3D data set includes information extracted from said 3D data set.

42. A method according to claim 41, wherein said information comprises at least one surface.

43. A method according to claim 41, wherein said information comprises at least one tissue identification.

44. A method according to claim 41, wherein said information comprises at least one bend or bifurcation location.

45. A method according to claim 33, wherein collecting comprises at least two of (i)-(iii).

46. A method according to claim 33, wherein collecting comprises all three of (i)-(iii).

47. A method according to claim 33, comprising cleaning up said collected 3D data set.

48. A method according to claim 47, wherein cleaning up comprises removing tissue indications belonging to a vessel other than a vessel of interest.

49. Apparatus comprising:
(a) a data input for x-ray projection data from an x-ray detector;
(b) a data input for IVUS data from an IVUS imager;
(c) a display; and
(d) a controller adapted and configured to generate a display using said IVUS data, based on a single projection from said detector.

50. Apparatus according to claim 49, comprising a data input for a 3D data set and wherein said controller is adapted to use said data set for generating said display.

51. Apparatus according to claim 49, wherein said controller is adapted to use multiple projection images.

52. Apparatus according to claim 49, wherein said apparatus comprises a C-arm imager.
